(19) United States
(12) Patent Application Publication
(10) Pub. No.: US 2024/0065538 A1
(43) Pub. Date: Feb. 29, 2024

(54) ENDOSCOPIC LENS CLEANING IN A ROBOTIC SURGICAL PROCEDURE

(71) Applicant: Titan Medical Inc., Toronto (CA)

(72) Inventors: Alan G. Sharer, Garner, NC (US); Antony R. Burness, Cambridge (GB); Clodagh E. Hogan, Cambridge (GB); Leigh R. Shelford, Cambridge (GB); Paul Smitheman, Cambridge (GB); Aki Hannu Einari Laakso, Raleigh, NC (US); Hans Christian Pflaumer, Apex, NC (US)

(21) Appl. No.: 17/895,369

(22) Filed: Aug. 25, 2022

(51) Int. Cl. A61B 1/12; A61B 1/00; A61B 1/005; A61B 34/37

(52) U.S. Cl. CPC A61B 1/126; A61B 1/00006; A61B 1/005; A61B 34/37; A61B 2034/301

(57) ABSTRACT

A method for cleaning an endoscopic instrument of a robotic surgical system includes, in response to receiving an input to effectuate a cleaning of the endoscopic instrument, robotically retracting the endoscopic instrument from a patient; and robotically moving an elongated shaft of the endoscopic instrument, relative to a longitudinal axis of the endoscopic instrument, from an unarticulated position to an articulated position after the elongated shaft of the endoscopic instrument is fully withdrawn from the patient.

### TECHNICAL FIELD

This disclosure relates to robotic surgical systems and, more particularly, to systems and methods for cleaning an endoscopic lens during a surgical procedure.

### BACKGROUND

Robotic surgical systems include control drive assemblies supporting surgical instruments used in laparoscopic and/or robotic surgery. These surgical instruments generally have a proximally located actuating mechanism that is operably coupled to a control drive unit of the control drive assembly for actuating distal end effectors of the surgical instruments. The control drive unit includes any number of motors operably associated with the actuating mechanisms of the surgical instruments. A clinician remotely controls these motors to enable the surgical instruments to robotically perform a surgical task within a body cavity of a patient, and often in remote locations within the body cavity that are not easily accessed without robotic surgical systems. Often during the procedure, an endoscopic lens of an endoscope of the robotic surgical system gets dirty and requires removal for cleaning. Such cleaning typically requires the endoscope to be completely removed from the robotic surgical system.

### SUMMARY

According to an aspect of this disclosure, a method for cleaning an endoscopic instrument of a robotic surgical system is provided. The method includes, in response to receiving an input to effectuate a cleaning of the endoscopic instrument, robotically retracting the endoscopic instrument from a patient; and robotically moving an elongated shaft of the endoscopic instrument, relative to a longitudinal axis of the endoscopic instrument, from an unarticulated position to an articulated position after the elongated shaft of the endoscopic instrument is fully withdrawn from the patient.

In an aspect, the elongated shaft includes a distal portion, and the method further includes moving the distal portion of the elongated shaft relative to a proximal portion of the elongated shaft.

The method may further include robotically moving the elongated shaft of the endoscopic instrument after the distal portion of the elongated shaft is withdrawn from a surgical port assembly.

The method may still further include receiving an input from a control drive unit that is configured to support and drive the endoscopic instrument.

The endoscopic instrument may be an endoscope. In an aspect, the method may further include articulating the distal portion of the endoscope to present a distal lens of the endoscope for cleaning.

The method may further include, in response to receiving an input, robotically moving the distal portion of the endoscopic instrument from an angled position, relative to the longitudinal axis, to a position that is coaxial with the longitudinal axis.

In an aspect, the method includes robotically inserting the endoscopic instrument distally back through the surgical portal assembly and into the patient after the distal portion is moved to the coaxial position.

In a further aspect, robotically retracting the endoscopic instrument may be caused by a receipt of an input signal from a surgeon console.

In another aspect, robotically moving the distal portion of the elongated shaft relative to the proximal portion of the elongated shaft may include pitching the distal portion relative to the proximal portion.

Robotically retracting the endoscopic instrument may include axially retracting an endoscopic drive unit of the control drive unit into the control drive unit.

According to another aspect of this disclosure, a robotic surgical system is provided and includes a control drive unit; an endoscope drive unit; an endoscopic instrument selectively attachable to the endoscope drive unit, the endoscopic instrument defining a longitudinal axis and including an elongated shaft supporting a distal lens; and a control pad coupled to the control drive unit, the control pad including a cleaning button, the cleaning button being configured to cause the elongated shaft to move relative to the longitudinal axis from an insertion position to a cleaning position, in response to an actuation of the cleaning button.

A distal portion of the elongated shaft may be movable relative to a proximal portion of the elongated shaft, when a longitudinal axis of the distal portion is angled relative to a longitudinal axis of the proximal portion.

The distal portion may be coaxial with the proximal position when the elongated shaft is in the insertion position.

The control pad may include an endoscope translation button that is operably coupled to the endoscope drive unit and configured to cause the endoscope drive unit to axially translate relative to the control drive unit in response to an actuation of the endoscope translation button.

When the endoscopic instrument is disposed in a retracted position upon actuation of the endoscope translation button, the endoscope drive unit may be distally advanced out of the control drive unit for advancing the endoscopic instrument through a surgical portal assembly and into a patient.

The control drive unit may further include a support bar assembly that supports the surgical portal assembly.

The support bar assembly may support the control pad on a first side of the support bar assembly.

The support bar assembly may support a second control pad on a second side of the support bar assembly.

The second control pad may have a second cleaning button that is actuatable to control the endoscopic instrument.

According to yet another aspect of this disclosure, a method for cleaning a distal portion of an endoscope of a robotic surgical system is provided. The method includes, in response to receiving an activation signal from an activation button, to effectuate a cleaning of the endoscope, robotically articulating a distal portion of an elongated shaft of the endoscope relative to a proximal portion of the endoscope, wherein a longitudinal axis of the distal portion is coaxial with a longitudinal axis of the proximal portion; retracting the endoscope from a patient and from a surgical port assembly such that a distal lens of the distal portion of the endoscope is withdrawn and free from the surgical port assembly; articulating the distal portion of the elongated shaft of the endoscope relative to the proximal portion of the endoscope, wherein the longitudinal axis of the distal portion is angled relative to the longitudinal axis of the proximal portion, and wherein the distal portion is oriented to facilitate a cleaning of the distal lens; articulating the distal portion of an elongated shaft of the endoscope relative to the proximal portion of the endoscope, wherein the longitudinal axis of the distal portion is again coaxial with the longitudinal axis of the proximal portion; and reinserting the endoscope into the patient, through the surgical port assembly.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIG. 2 is an enlarged, front, perspective view of a control drive assembly of the robotic surgical system of FIG. 1; and

FIGS. 3-10 are progressive views illustrating a lens of an endoscope of the robotic surgical system being cleaned in accordance with the principles of this disclosure.

### DETAILED DESCRIPTION

Aspects of this disclosure are described in detail with reference to the drawings, in which like reference numerals designate identical or corresponding elements in each of the several views. As used herein, the term “distal” refers to that portion of structure closer to a patient, while the term “proximal” refers to that portion of structure, farther from the patient. As used herein, the term “clinician” refers to a doctor, nurse, or other care provider and may include support personnel and/or equipment operators.

In the following description, well-known functions or constructions are not described in detail to avoid obscuring the present disclosure in unnecessary detail.

Robotic surgical systems have been used in minimally invasive medical procedures. Such procedures may be referred to as what is commonly referred to as “Telesurgery.” These robotic surgical systems have one or more surgical instruments removably coupled thereto. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robotic surgical system for selectively operating end effectors of the connected surgical instruments.

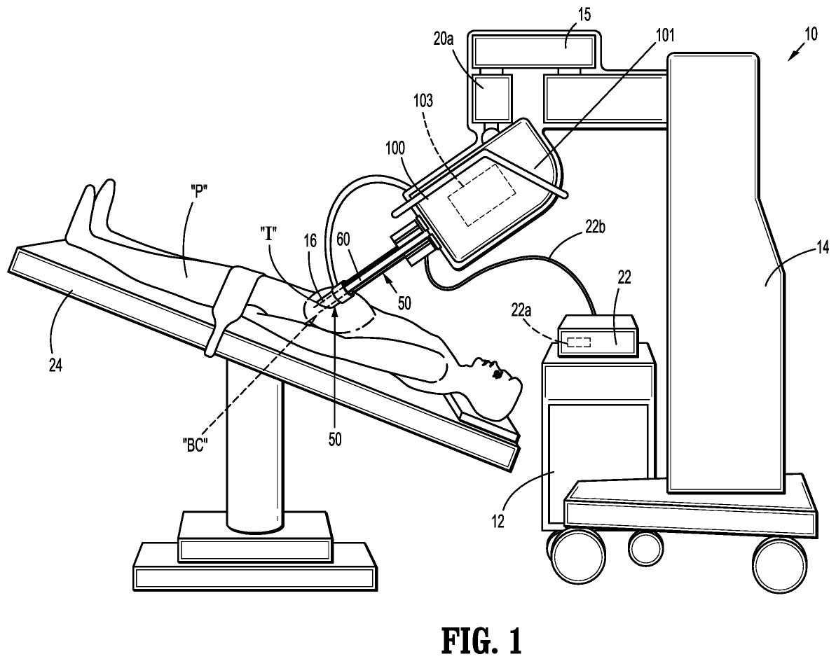

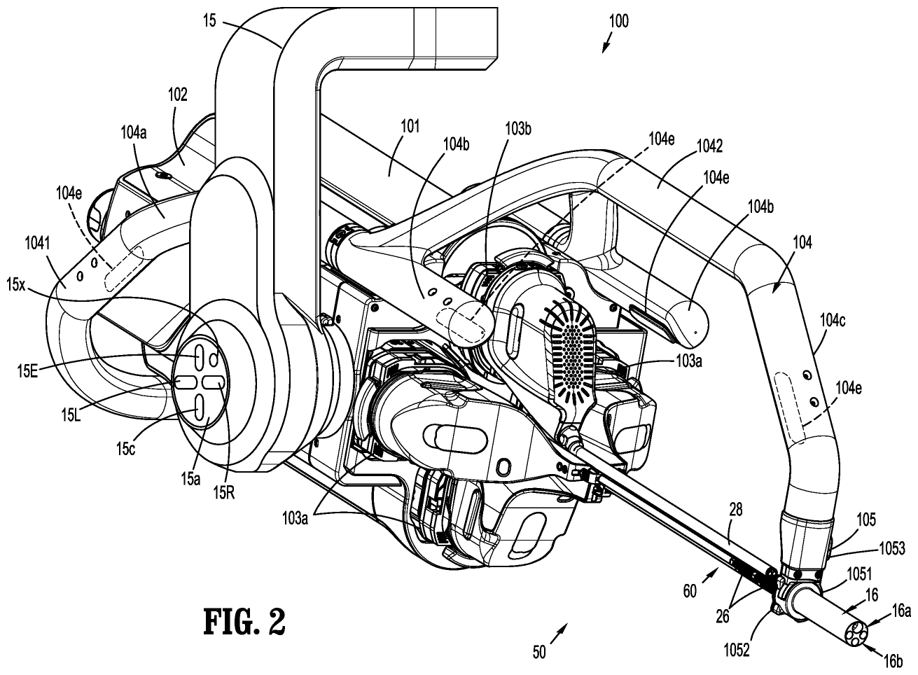

With reference to FIGS. 1 and 2, a robotic surgical system is shown generally at 10. Robotic surgical system 10 employs various robotic elements to assist the clinician and allow remote operation (or partial remote operation) of surgical instruments 60 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. The instrument cart 14 supports a control drive assembly 100 on a setup arm assembly 15 that is selectively movable relative to instrument cart 14. Control drive assembly 100 includes one or more surgical instrument systems 50 mounted on a control drive unit 101 supported on setup arm assembly 15. Control drive unit 101 is movable relative to cart 14 and houses an instrument drive assembly 103 for manipulating the surgical instrument systems 50 and/or independent surgical instruments 60 thereof with the assistance of, for example one or more computing devices or controllers. Instrument drive assembly 103 can include an instrument drive unit 103a for operating surgical devices such as graspers coupled thereto and an endoscope drive unit 103b for operating surgical devices such as an endoscope coupled thereto. Surgical instrument system 50 can include any number and/or type of surgical instruments. The surgical instruments 60 can include, for example, graspers or forceps 26, which may be electrosurgical, an endoscope 28, and/or any other suitable instrument that can be driven by one or more associated tool drives, such as instrument drive unit 103a and endoscope drive unit 103b of instrument drive assembly 103. For example, besides graspers 26 and endoscope 28, the one or more surgical instruments 60 can include dexterous tools, such as grippers, needle drivers, staplers, dissectors, cutters, hooks, graspers, scissors, coagulators, irrigators, suction devices, which are used for performing a surgical procedure.

With reference to FIGS. 1 and 2, surgical instrument system 50 further includes a surgical portal assembly 16 defining a plurality of separate and spaced-apart conduits, channels or lumens 16a, 16b therethrough that are configured to receive, for instance, the surgical instruments 60 for accessing a body cavity “BC” of a patient “P.” Such lumens 16a, 16 may be of any suitable shape and/or size. In other aspects, the surgical portal assembly 16 may define a single conduit, channel, or lumen therethrough that is configured to receive, for instance, the surgical instruments 60 for accessing a body cavity “BC” of a patient “P.” In particular, the surgical portal assembly 16 can be inserted through an incision “I” and into the body cavity “BC” of the patient “P”.

With reference again to FIG. 1, the workstation 12 includes an input device 22 in communication with control drive unit 101 for use by a clinician to control the surgical portal assembly 16 and the various surgical instrument systems 50 (and surgical instruments 60 thereof) via the instrument drive assembly 103 for performing surgical operations on the patient “P” while the patient “P” is supported on a surgical table 24, for example. Input device 22 is configured to receive input from the clinician and produces input signals. Input device 22 may also be configured to generate feedback to the clinician. The feedback can be visual, auditory, haptic, or the like.

The workstation 12 can further include computing devices and/or controllers such as a master processor circuit 22a in communication with the input device 22 for receiving the input signals and generating control signals for controlling the robotic surgical system 10, which can be transmitted to the instrument cart 14 via an interface cable 22b. In some cases, transmission can be wireless and interface cable 22b may not be present. The input device 22 can include right and left-hand controls (not shown) and/or foot pedals (not shown), which are moved/operated to produce input signals at the input device 22 and/or to control robotic surgical system 10. The instrument cart 14 can include a slave processor circuit 20a that receives and the control signals from the master processor circuit 22a and produces slave control signals operable to control the various surgical instrument systems 50 (and surgical instruments 60 thereof) during a surgical procedure. The workstation 12 can also include a user interface, such as a display (not shown) in communication with the master processor circuit 22a for displaying information (such as, body cavity images) for a region or site of interest (for example, a surgical site, a body cavity, or the like) and other information to a clinician. While both master and slave processor circuits are illustrated, in other aspects, a single processor circuit may be used to perform both master and slave functions.

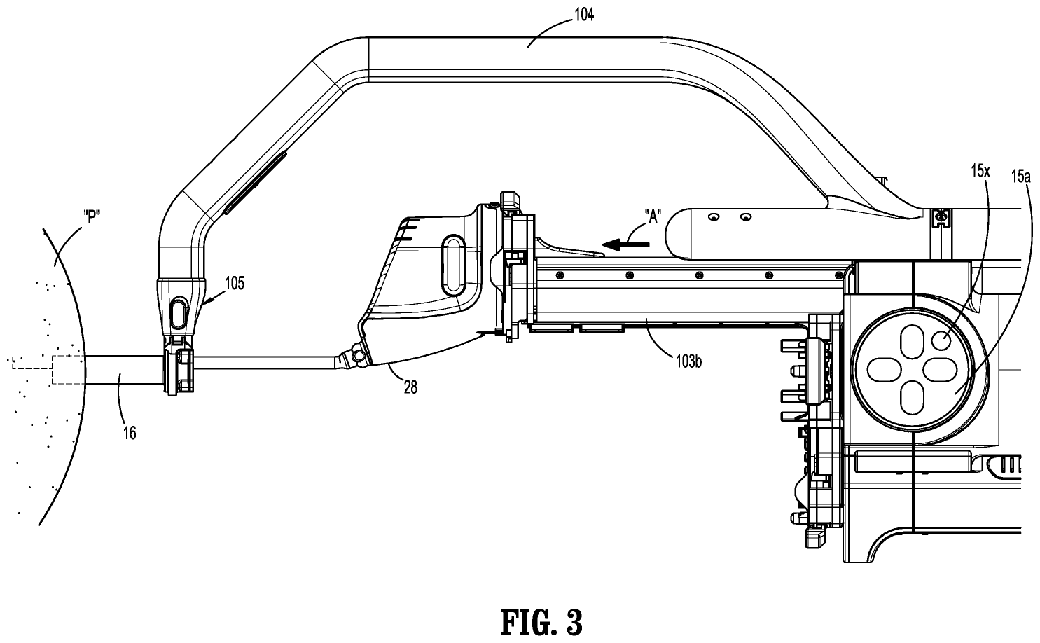

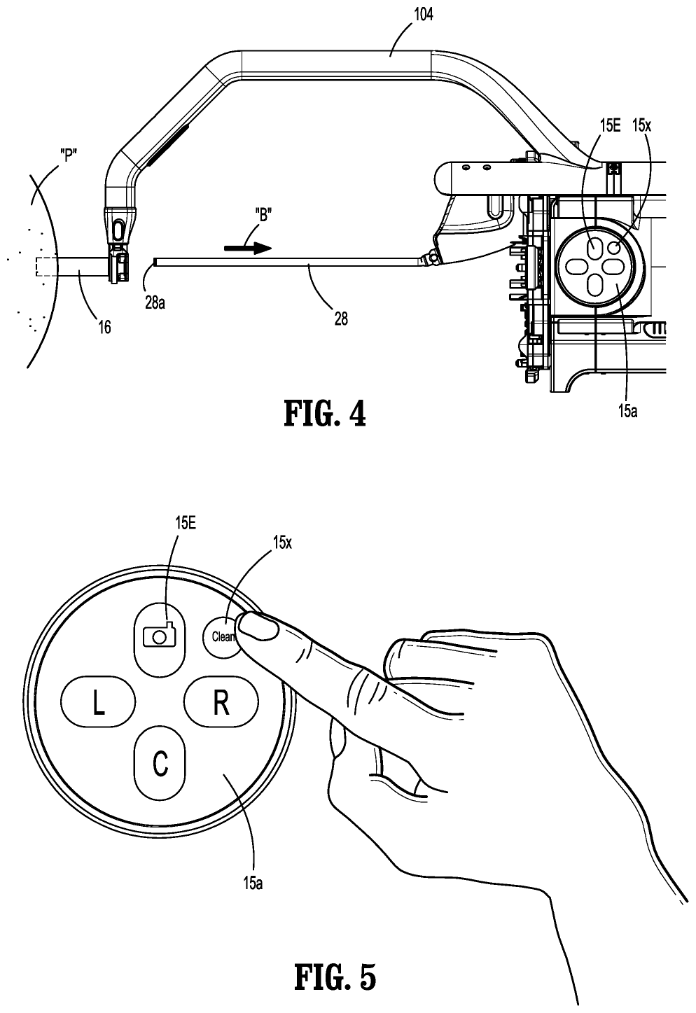

With reference to FIGS. 1 and 2, control drive assembly 100 is pivotably mounted to setup arm assembly 15. Control drive unit 101 of control drive assembly 100 includes a housing 102 that supports instrument drive assembly 103, including instrument drive units 103a (e.g., three) and endoscope drive unit 103b. Control drive unit 101 further includes a support bar assembly 104 that is mounted to housing 102. Setup arm assembly 15 includes a first control pad 15a having buttons 15L, 15R, 15C, and 15E that are selectively actuatable to cause linear translation of respective ones of instrument drive and endoscope drive units 103a, 103b relative to control drive unit 101 as indicated by arrows “A” and “B” shown in FIGS. 3 and 4, respectively. First control pad 15a further includes a cleaning button 15x. Housing 102 also supports a second control pad (not explicitly shown) that is on the opposite side of setup arm assembly 15 as first control pad 15a but similarly includes buttons 15L, 15R, 15C, 15E, and 15x that likewise provides the same functions as the corresponding buttons on the first control pad 15a. Support bar assembly 104 includes a first portion 1041 including a rear bar 104a having a U-shaped configuration that extends around sidewalls of a proximal portion of housing 102 and beneath housing 102, and a second portion 1042 having handles 104b, and a port arm 104c that extends from handles 104b distally to port latch assembly 105 on a distal end portion of port arm 104c. Port latch assembly 105 includes a pivotable clamp arm 1051, a fixed clamp arm 1052, and an actuator 1053 operatively coupled to pivotable clamp arm 1051 to cause pivotable clamp arm 1051 to selectively couple to surgical portal assembly 16. Support bar assembly 104 further includes a plurality of brake release buttons 104e on inner facing surfaces of rear bar 104a, handles 104b, and port arm 104c of support bar assembly 104 that are configured to stop linear movement of instrument drive and/or endoscope drive units 103a, 103b relative to control drive unit 101.

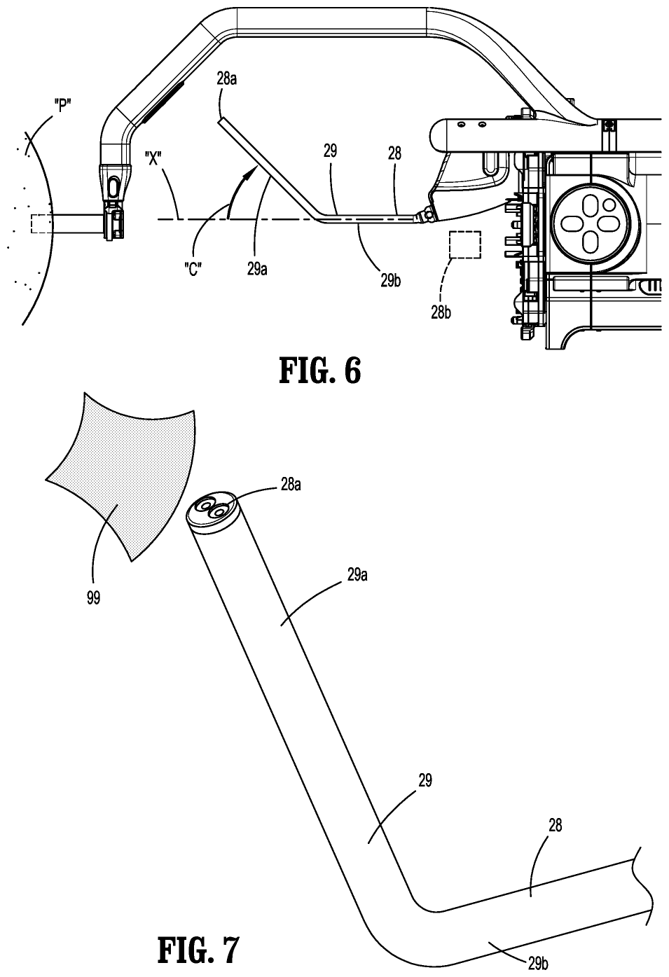

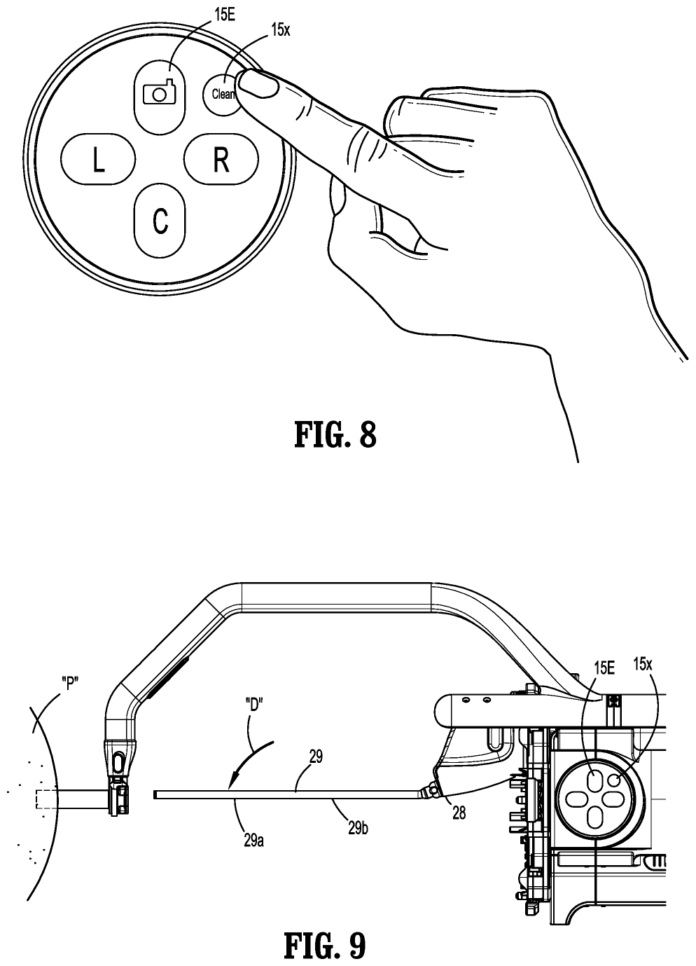

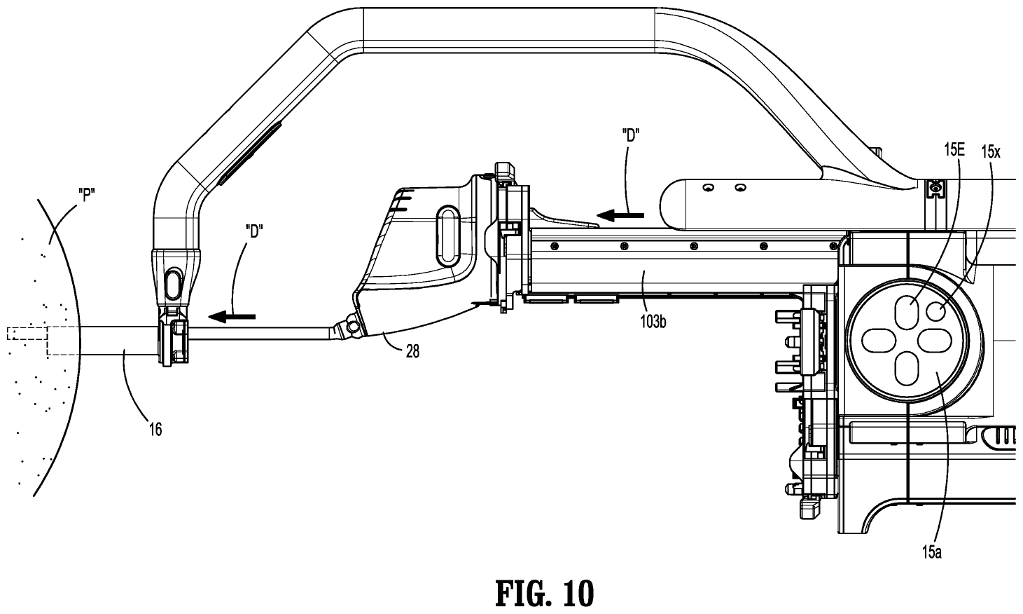

Turning now to FIGS. 3-10, endoscope 28 defines a longitudinal axis “X” and includes a distal lens 28a (or a plurality of distal lenses) and a drive assembly 28b. Endoscope 28 further includes an elongated shaft 29 having a distal portion 29a and a proximal portion 29b. Distal portion 29a is operatively coupled to drive assembly 28b to enable drive assembly 28b to move (e.g., articulate) distal portion 29a relative to proximal portion 29b and relative to longitudinal axis “X.” Distal lens 28a is supported on a distal end of distal portion 29 of elongated shaft 29.

During a surgical procedure, distal lens 28a of endoscope 28 can get dirty with, for example, tissue, blood, or other fluid and/or debris from the surgical procedure. When a clinician determines that such distal lens 28a needs to be cleaned, the endoscope 28 is axially retracted from the patient “P” along longitudinal axis “X” and out of the surgical portal assembly 16 such that endoscope drive unit 103b retracts into housing 102 of control drive unit 101 upon an actuation of endoscope translation button 15E. The cleaning button 15x can then be manually actuated, which causes a drive assembly 28b of endoscope 28, in response to an input signal received by control drive assembly 100, control drive unit 101, instrument drive assembly 103 and/or endoscope drive assembly 28b, to articulate (e.g., pitch) distal portion 29a of elongated shaft 29 of endoscope 28 relative to proximal portion 29b of elongated shaft 29, as indicated by arrow “C.” In particular, distal portion 29a can pitch and/or yaw upwardly, downwardly, and/or laterally from a coaxially position relative to a longitudinal axis “X” of endoscope 28 to an angled or offset position relative to the longitudinal axis “X” such that distal portion 29a is disposed an angle of orientation relative to proximal portion 29b. The angle of orientation is configured to be sufficiently large enough to present distal lens 28a in a position that can readily be accessed by a clinician for wiping distal lens 28a, for instance, with a sterile sponge or cloth 99 to clean distal lens 28a. For example, the angle may be between about 10 degrees to about 90 degrees relative to the longitudinal axis “X” in any direction, and may be up to about 180 degrees in any direction. Drive assembly 28b of endoscope 28 can have any suitable electromechanical structures to facilitate the articulating movement of elongated shaft 29. For instance, drive assembly 28b and elongated shaft 29 may include and/or support cables, gears, pulleys, etc. that cooperate to effectuate articulating/pivoting movement of distal portion 29a relative to proximal portion 29b.

Once distal lens 28a of endoscope 28 is determined by the clinician to be sufficiently clean, the clinician can again actuate the cleaning button 15x, and in response to an input signal received by control drive assembly 100, control drive unit 101, instrument drive assembly 103 and/or endoscope drive assembly 28b, to cause the distal portion 29a to return to the initial position, as indicated by arrows “D,” in which the distal portion 29a is coaxial with the proximal portion 29b. The endoscope 28 can then be inserted back through surgical portal assembly 16 and into patient “P” upon another actuation of endoscope translation button 15E for continuing the surgical procedure. This process can be repeated as needed before, during, and/or after a surgical procedure.

The disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing devices (and/or servers) can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from camera), accessing storage device of the controller, generating a raw image based on the sensed data, comparing the raw image to a calibration data set, identifying an object based on the raw image compared to the calibration data set, transmitting object data to a post-processing unit, and displaying the object data to a graphic user interface. Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” “in other aspects” or the like may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to all such states. Reference to a program may encompass the actual instructions and/or the intent of those instructions.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A method for cleaning an endoscopic instrument of a robotic surgical system, the method comprising:
in response to receiving an input to effectuate a cleaning of the endoscopic instrument, robotically retracting the endoscopic instrument from a patient; and
robotically moving an elongated shaft of the endoscopic instrument, relative to a longitudinal axis of the endoscopic instrument, from an unarticulated position to an articulated position after the elongated shaft of the endoscopic instrument is fully withdrawn from the patient.

2. The method of claim 1, wherein the elongated shaft includes a distal portion, the method further comprising moving the distal portion of the elongated shaft relative to a proximal portion of the elongated shaft.

3. The method of claim 2, further comprising robotically moving the elongated shaft of the endoscopic instrument after the distal portion of the elongated shaft is withdrawn from a surgical port assembly.

4. The method of claim 2, further comprising receiving an input from a control drive unit that is configured to support and drive the endoscopic instrument.

5. The method of claim 2, wherein the endoscopic instrument is an endoscope, and wherein the method includes articulating the distal portion of the endoscope to present a distal lens of the endoscope for cleaning.

6. The method of claim 3, further comprising, in response to receiving an input, robotically moving the distal portion of the endoscopic instrument from an angled position, relative to the longitudinal axis, to a position that is coaxial with the longitudinal axis.

7. The method of claim 6, further comprising robotically inserting the endoscopic instrument distally back through the surgical portal assembly and into the patient after the distal portion is moved to the coaxial position.

8. The method of claim 1, wherein robotically retracting the endoscopic instrument is caused by a receipt of an input signal from a surgeon console.

9. The method of claim 2, wherein robotically moving the distal portion of the elongated shaft relative to the proximal portion of the elongated shaft includes pitching the distal portion relative to the proximal portion.

10. The method of claim 1, wherein robotically retracting the endoscopic instrument includes axially retracting an endoscopic drive unit of the control drive unit into the control drive unit.

11. A robotic surgical system comprising:
a control drive unit;
an endoscope drive unit;
an endoscopic instrument selectively attachable to the endoscope drive unit, the endoscopic instrument defining a longitudinal axis and including an elongated shaft supporting a distal lens; and
a control pad coupled to the control drive unit, the control pad including a cleaning button, the cleaning button being configured to cause the elongated shaft to move relative to the longitudinal axis from an insertion position to a cleaning position, in response to an actuation of the cleaning button.

12. The robotic surgical system of claim 11, wherein a distal portion of the elongated shaft is movable relative to a proximal portion of the elongated shaft, when a longitudinal axis of the distal portion is angled relative to a longitudinal axis of the proximal portion.

13. The robotic surgical system of claim 12, wherein the distal portion is coaxial with the proximal position when the elongated shaft is in the insertion position.

14. The robotic surgical system of claim 11, wherein the control pad includes an endoscope translation button that is operably coupled to the endoscope drive unit and configured to cause the endoscope drive unit to axially translate relative to the control drive unit in response to an actuation of the endoscope translation button.

15. The robotic surgical system of claim 14, wherein, when the endoscopic instrument is disposed in a retracted position upon actuation of the endoscope translation button, the endoscope drive unit is distally advanced out of the control drive unit for advancing the endoscopic instrument through a surgical portal assembly and into a patient.

16. The robotic surgical system of claim 15, wherein the control drive unit further includes a support bar assembly that supports the surgical portal assembly.

17. The robotic surgical system of claim 16, wherein the support bar assembly supports the control pad on a first side of the support bar assembly.

18. The robotic surgical system of claim 17, wherein the support bar assembly supports a second control pad on a second side of the support bar assembly.

19. The robotic surgical system of claim 18, wherein the second control pad has a second cleaning button that is actuatable to control the endoscopic instrument.

20. A method for cleaning a distal portion of an endoscope of a robotic surgical system, the method comprising:
in response to receiving an activation signal from an activation button, to effectuate a cleaning of the endoscope, robotically:
articulating a distal portion of an elongated shaft of the endoscope relative to a proximal portion of the endoscope, wherein a longitudinal axis of the distal portion is coaxial with a longitudinal axis of the proximal portion;
retracting the endoscope from a patient and from a surgical port assembly such that a distal lens of the distal portion of the endoscope is withdrawn and free from the surgical port assembly;
articulating the distal portion of the elongated shaft of the endoscope relative to the proximal portion of the endoscope, wherein the longitudinal axis of the distal portion is angled relative to the longitudinal axis of the proximal portion, and wherein the distal portion is oriented to facilitate a cleaning of the distal lens;
articulating the distal portion of an elongated shaft of the endoscope relative to the proximal portion of the endoscope, wherein the longitudinal axis of the distal portion is again coaxial with the longitudinal axis of the proximal portion; and
reinserting the endoscope into the patient, through the surgical port assembly.
